(19) United States
(12) Patent Application Publication
(10) Pub. No.: US 2018/0206722 A1
(43) Pub. Date: Jul. 26, 2018

(54) MEDICAL DATA BRACELET

(71) Applicant: Melissa Gamez, Ayer, MA (US)

(72) Inventor: Melissa Gamez, Ayer, MA (US)

(21) Appl. No.: 15/412,331

(22) Filed: Jan. 23, 2017

(51) Int. Cl. A61B 5/00; A44C 5/00; A61B 5/0205; A61B 5/1455

(52) U.S. Cl. CPC A61B 5/0022; A44C 5/0015; A44C 5/0023; A61B 5/02438; A61B 5/14552; A61B 5/681; A61B 5/02055

(57) ABSTRACT

A system for vital sign data management and transmission that includes: a bracelet, wherein the bracelet includes a plurality of vital sign sensors; a network connection and a caregiver portable electronic device. The bracelet is adapted to connect to the network connection to transmit vital sign data to a central database and processor. The caregiver uses a portable electronic device to receive the vital sign data via a software application which interacts with the central database and processor via the network connection.

### BACKGROUND OF THE INVENTION

### Field of Invention

The present invention relates to an electronic bracelet used to monitor vital signs of a user.

### Description of Related Art

Currently there are various health monitoring devices that are available for users for various purposes. One common device relates to emergency alert systems, where a user may wear a necklace have an easy, convenient alerting device available that alerts need for medical attention. These alert systems are commonly used with elderly individuals who may live alone. Such systems are particularly helpful should the elderly person fall or becomes immobile, they may easily provide an alert for assistance. Other vital monitoring systems have been developed that relate to physical activity. These systems are commonly worn as watches or wrist bracelets that monitor heart rate, steps and other physical activity. It would be advantageous to have such a system that combined aspects from both of these applications. In particular a bracelet that may be worn to monitor heart rate and other vital signs that may be electronically relayed to a remote caregiver.

### SUMMARY OF THE INVENTION

The present invention relates to a system for vital sign data management and transmission that includes: a bracelet, wherein the bracelet includes a plurality of vital sign sensors; a network connection and a caregiver portable electronic device. The bracelet is adapted to connect to the network connection to transmit vital sign data to a central database and processor. The caregiver uses a portable electronic device to receive the vital sign data via a software application which interacts with the central database and processor via the network connection.

### BRIEF DESCRIPTION OF DRAWINGS

FIG. 1 depicts an alert bracelet in accordance with the present invention.

### DETAILED DESCRIPTION

The present invention relates to a bracelet is worn and particularly preferably by elderly individuals to monitor vital signs. The present invention includes sensors for pulse, blood oxygen and blood pressure readings. The readings may be displayed on a screen provided on the bracelet. Further, the bracelet includes wireless transmission capabilities so that the bracelet transmits data via a network to a central database. The database transfers the information to a remote caregiver, so the remote caregiver may receive constant vital sign information regarding a user of the bracelet. Preferably, the caregiver receives this information through a smart phone application that connects to a central database and processor. As a result, caregivers will be alert to any abnormal vital sign readings that are transmitted periodically from the bracelet.

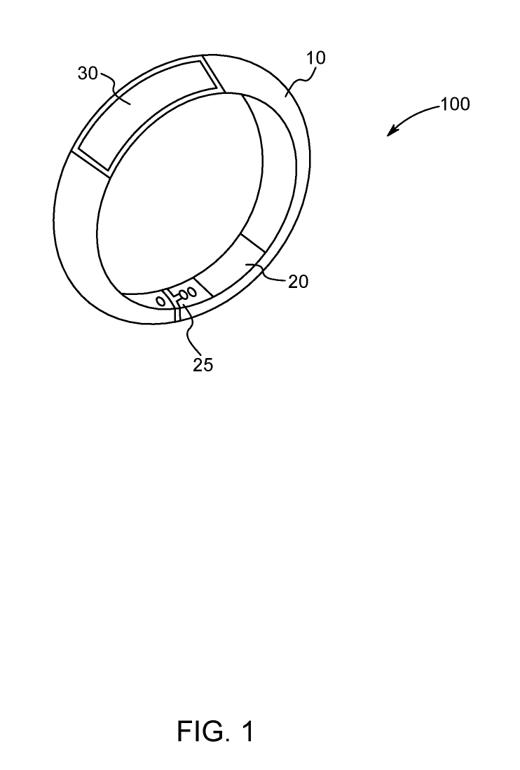

In reference to FIG. 1, a Medical Bracelet 100 in accordance with the present invention is depicted. The Medical Bracelet 100 includes a clasp 25 and a series of sensors 20 on the inner surface of the bracelet 100. The sensors 20 on the bracelet 100 may include pulse sensor, thermometer and oxygen readings through a oximeter. These readings may be viewed on a screen 30 provided on the outer surface of the Bracelet 100. The screen 30 may depict these readings periodically or on demand. The Bracelet 100 also includes wireless transmissions through a network where the network receives data compiled from the bracelet readings. This data may be transmitted over the network to an end user via the end user's portable electronic device such as a smart phone, tablet or laptop computer. If using a smart phone or tablet, an application receives this data on a periodic basis therefore providing the caregiver updated vital signs as needed. The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed:

1. A system for vital sign data management and transmission comprising:
a bracelet, wherein the bracelet includes a plurality of vital sign sensors;
a network connection, wherein the bracelet is adapted to connect to the network connection;
a central database and processor, wherein the central database and processor receives data from the bracelet via the network connection; and
a portable electronic device, wherein the portable electronic device includes a software application to interact with the central database and processor via the network connection.

2. The system of vital sign date management according to claim 1, where the plurality of vital sign sensors include at least a pulse sensor, a thermometer and an oximeter.

3. The system of vital sign data management according to claim 1, where the portable electronic device includes at least a smartphone, a tablet or a laptop computer.
